SEQUENCE LISTING (18 sequences)

SEQ ID NO: 1 - DNA, 600 bp, Saccharomyces cerevisiae
agaaaacatagaatagttaccgttattggtaggagtgtggtggggtggtatagtccgcattgggatgttactttcctgttatggcatggatttccctttagggtctctgaagcgtatttccgtcaccgaaaaaggcagaaaaagggaaactgaagggaggatagtagtaaagtttgaatggtggtagtgtaatgtatgatatccgttggttttggtttcggttgtgaaaagttttttggtatgatattttgcaagtagcatatatttcttgtgtgagaaaggtatattttgtatgttttgtatgttcccgcgcgtttccgtattttccgcttccgcttccgcagtaaaaaatagtgaggaactgggttacccggggcacctgtcactttggaaaaaaaatatacgctaagatttttggagaatagcttaaattgaagtttttctcggcgagaaatacgtagttaaggcagagcgacagagagggcaaaagaaaataaaagtaagattttagtttgtaatgggagggggggtttagtcatggagtacaagtgtgaggaaaagtagttgggaggtacttcatgcgaaagcagttgaagacaa

SEQ ID NO: 2 - DNA, 284 bp, Saccharomyces cerevisiae
tttttatttctttctaagtgggtactggcaggagccggggcctagtttagagagaagtagactgaacaagtctctataaattttatttgtcttaagaattctatgatccgggtaaaaacatgtattgtatatatctattataatatacgatgaggatgatagtgtgtaagagtgtaccatttactaatgtatgtaagttactatttactatttggtctttttattttttattttttttttttttttcgttgcaaagatgggttgaaagagaagggctttcacaa

SEQ ID NO: 3 - DNA, 180 bp, Saccharomyces cerevisiae
aaagcaaaaatgtgatcttgcttgtaaatacaattttgagaggttaataaattacaagtagtgctatttttgtatttaggttagctatttagctttacgttccaggatgcctagtggcagccccacaatatccaggaagccctctctgcggtttttcagattaggtagtcgaaaaaccta

SEQ ID NO: 4 - DNA, 1020 bp, Saccharomyces cerevisiae
atgcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaagga

SEQ ID NO: 5 - DNA, 50 bp, Artificial Sequence
  Poly (T) sequence
tttttttttttttttttttttttttttttttttttttttttttttttttt

SEQ ID NO: 6 - DNA, 804 bp, Streptomyces hygroscopicus
atgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaa

SEQ ID NO: 7 - DNA, 810 bp, Micromonospora rhodorangea
atgggtaaggaaaagactcacgtttcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaa

SEQ ID NO: 8 - DNA, 344 bp, Saccharomyces cerevisiae
gacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaacc

SEQ ID NO: 9 - DNA, 198 bp, Saccharomyces cerevisiae
actgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactg

SEQ ID NO: 10 - DNA, 50 bp, Artificial Sequence
  Primer
catttccccgaaaagtgccacctgacgtcgacatggaggcccagaatacc

SEQ ID NO: 11 - DNA, 51 bp, Artificial Sequence
  Primer
ctttagcggcttaactgtgccctccatggcagtatagcgaccagcattcac

SEQ ID NO: 12 - DNA, 18 bp, Artificial Sequence
  Primer
atgcctgaactcaccgcg

SEQ ID NO: 13 - DNA, 51 bp, Artificial Sequence
  Primer
gaaagcagttgaagacaagttcgaaaagagaaagcaaaaatgtgatcttgc

SEQ ID NO: 14 - DNA, 33 bp, Artificial Sequence
  Primer
ttggcgcgccttgaaatgtagcaggtaaatttc

SEQ ID NO: 15 - DNA, 31 bp, Artificial Sequence
  Primer
ttggcgcgccatgtcgaaagctacatataag

SEQ ID NO: 16 - DNA, 26 bp, Artificial Sequence
  Primer
ccgctcgagttagttttgctggccgc

SEQ ID NO: 17 - DNA, 35 bp, Artificial Sequence
  Primer
ctagtctagatttttatttctttctaagtgggtac

SEQ ID NO: 18 - DNA, 30 bp, Artificial Sequence
  Primer
gatgctagcttgtgaaagcccttctctttc

(19) United States
(12) Patent Application Publication
(10) Pub. No.: US 2019/0225973 A1
(43) Pub. Date: Jul. 25, 2019

(54) NOVEL SACCHAROMYCES CEREVISIAE EXPRESSION SYSTEM AND CONSTRUCTION METHOD THEREOF

(71) Applicant: Qilu University of Technology, Jinan (CN)

(72) Inventors: Xiaoming Bao, Jinan (CN); Lili Xu, Jinan (CN); Chenxi Qiu, Jinan (CN); Hongxing Li, Jinan (CN); Yong Yi, Jinan (CN); Jixiang Zhang, Jinan (CN); Chuanchao Fu, Jinan (CN); Dong Wang, Jinan (CN)

(21) Appl. No.: 16/252,934

(22) Filed: Jan. 21, 2019

(30) Foreign Application Priority Data
Jan. 24, 2018 (CN) 201810068015.X

(51) Int. Cl. C12N 15/81; C12N 15/65

(52) U.S. Cl. CPC C12N 15/81; C12N 15/65; C12N 2840/203; C12N 2830/001; C12N 2830/50

(57) ABSTRACT

A Saccharomyces cerevisiae expression system and a construction method and application thereof, including an expression vector which includes, from 5′ to 3′, a YEplac195 plasmid backbone, an exogenous gene expression cassette, and a selective marker gene expression cassette. The exogenous gene expression cassette includes from upstream to downstream an rDNA promoter, an internal ribosome entry site (IRES) sequence, an exogenous gene expression cassette, a poly(T) sequence, and an rDNA terminator. The selective marker gene expression cassette includes a promoter, a selective marker gene, and a transcription terminator.

### CROSS-REFERENCE TO RELATED APPLICATION

This application claims priority to Chinese application number 201810068015.X, filed Jan. 24, 2018, with a title of NOVEL SACCHAROMYCES CEREVISIAE EXPRESSION SYSTEM AND CONSTRUCTION METHOD THEREOF. The above-mentioned patent application is incorporated herein by reference in its entirety.

### TECHNICAL FIELD

The present invention relates to the field of biotechnology, and in particular to a Saccharomyces cerevisiae expression system and a construction method thereof.

### BACKGROUND

With the rapid development of genomics research, various expression systems such as bacteria, yeasts, insects, and mammalian cells have emerged at the right moment to meet the urgent needs of mining new genes and new functions thereof, constructing new engineering bacteria, and the like. There are many yeast expression systems, such as Saccharomyces cerevisiae, Schizosaccharomyces, Pichia pastoris, Kluyveromyces lactis, Candida utilis and the like, among which Saccharomyces cerevisiae and Pichia pastoris are the two most commonly used expression systems.

Saccharomyces cerevisiae, also known as baker's yeast, has long been used in wine making, production of bread and steamed buns, and the like. Saccharomyces cerevisiae is safe and reliable, does not produce toxins, and is a GRAS (Generally Regarded As Safe) eukaryotic microbe. Because its cells are rich in nutrients and have a high economic value, its yeast extract not only is widely used in cell culture for microorganisms, plants and animals and plays a decisive role in pharmaceuticals, brewing and fermented foods, but also is directly applied to feed and food additives. Further, because the yeast has a good fermentation performance in industrial production, can rapidly divide during fermentation, is easy to culture, has relatively strong resistance to microbial contamination, has a clear genetic background, and is simple to manipulate genetically, it is often used as the starting strain for metabolic engineering in genetic engineering technology. In synthetic biology research, Saccharomyces cerevisiae has become a high-profile chassis cell due to its metabolic capacity and other characteristics, which can be used for constructing microbial cell factories with different functions. Compared with prokaryotes, it can recognize and help transcription and post-translational modification of eukaryotic genes, express proteins with near-native conformations, and secrete heterologous proteins to the extracellular, which facilitates purification of expression products. Many genes involved in human genetic diseases have high homology with yeast genes, such that the yeast can also serve as a model organism for higher eukaryotes, especially for human genome research, to improve the level of gene diagnosis and treatment. As a result, the yeast expression system, in particular Saccharomyces cerevisiae, has become an important tool for production of bio-based chemicals, expression of novel exogenous genes, and for services in the fields of industry and agriculture, as well as medicine.

In normal conditions, like other eukaryotes, Saccharomyces cerevisiae produces at least three major RNAs, including ribosomal RNA (rRNA), transfer RNA (tRNA), and messenger RNA (mRNA), where rRNA is synthesized by RNA polymerase I. Responsible for synthesis, the code-expressed protein is synthesized by RNA polymerase II, and tRNA and 5S rRNA are synthesized by RNA polymerase III. The expression efficiency of foreign genes in the yeast is related to the strength of a promoter, the transcription efficiency of RNA polymerase II-regulated mRNAs, and like factors. The currently used Saccharomyces cerevisiae expression system lacks a strong promoter and is affected by other factors. Thus, it is difficult to express exogenous genes at high levels when fermentation production is conducted with Saccharomyces cerevisiae.

The rRNA produced by Saccharomyces cerevisiae accounts for 80% of the total RNA content [Warner J R. The economics of ribosome biosynthesis in yeast. Trends Biochem Sci. 1999, 24(10:437-440], which is assembled with ribosomal proteins and other related proteins in the nucleus to form large and small subunits, transferred out of the nucleus, assembled into mature ribosomes in the cytoplasm, to achieve protein translation. The yeast cell produces about 2,000 ribosomes per minute [Warner J R. The economics of ribosome biosynthesis in yeast. Trends Biochem Sci. 1999, 24(11):437-440.]. The rRNA-encoding rDNA gene is typically randomly located on chromosome XII in about 150-200 repeating unit copies [Petes T D: Yeast ribosomal DNA genes are located on chromosome XII. Proceedings of the National Academy of Sciences of the United States of America. 1979, 76(1):410-414], and transcription of the rDNA gene begins at the promoter site where an initial complex is formed from the RNA polymerase I and four major transcription factors, i.e., a core factor (CF), Rrn3p, a TATA binding protein (TBP), and an upstream activation factor (UAF). The energy input of the cell in biosynthesis of ribosome is greater than that of any other process. According to calculations, RNA polymerase I-mediated transcription initiation must occur every 5 s under standard yeast growth conditions [Reeder, R. H., Lang, W. H. Terminating transcription in eukaryotes: lessons learned from RNA polymerase I. Trends Biochem. 1997, Sci. 22:473-477]. The RNA polymerase I elongates through the 35S rRNA gene at approximately 60 nucleotides per second [French S L, Osheim Y N, Cioci F, Nomura M, Beyer A L. In exponentially growing Saccharomyces cerevisiae cells, ribosomal RNA synthesis is determined by the summed RNA polymerase I loading rate rather than by the number of active genes. Mol Cell Biol. 2003, 23:1558-1568]. The RNA polymerase I-mediated rDNA transcription is unique in terms of a high initiation rate, polymerase density, specific tissue within the nucleolus and tight connection to ribosome assembly, and accounts for more than 60% of the total nuclear transcription [Warner J R. The economics of ribosome biosynthesis in yeast. Trends Biochem Sci. 1999, 24(10:437-440]. The transcript has a full length of approximately 6.7 kb, which is also significantly longer than the products of transcriptions mediated by the RNA polymerases II and III. RNA polymerase I has transcription initiation and elongation efficiency that is significantly faster than those of the RNA polymerase II and the RNA polymerase III. Using the feature that the RNA polymerase I plays an efficient role in the transcription of the rDNA gene, we used an rDNA gene promoter to initiate the expression of an exogenous or endogenous gene, and thus construct a novel Saccharomyces cerevisiae expression system.

### SUMMARY

In view of the shortcomings of current yeast expression systems, a novel Saccharomyces cerevisiae expression system is constructed by using an rDNA gene promoter to initiate the expression of an exogenous or endogenous gene by means of the efficient function of RNA polymerase I in the transcription of an rDNA gene. Meanwhile, we also studied the RNA polymerase I-mediated regulatory mechanism and rRNA synthesis mechanism in Saccharomyces cerevisiae.

Various aspects of the present invention are described below.

A first aspect includes a Saccharomyces cerevisiae expression system that includes a host transfected by an expression vector, where the expression vector is circular and is a shuttle plasmid vector, and is constructed between Saccharomyces cerevisiae and Escherichia coli. The expression vector includes sequentially from 5′ to 3′ the following operable elements: a YEplac195 plasmid backbone, an exogenous or endogenous gene expression cassette, and a selective marker gene expression cassette. The YEplac195 plasmid is a yeast episomal plasmid, which contains the ori of the yeast 2μ plasmid. The exogenous or endogenous gene expression cassette includes sequentially from upstream to downstream: an rDNA promoter, an internal ribosome entry site (IRES) sequence, an exogenous or endogenous gene expression cassette, a poly(T) sequence, and an rDNA terminator. And the selective marker gene expression cassette includes a promoter, a selective marker gene, and a transcription terminator.

An open reading frame in the exogenous or endogenous gene expression cassette is a uracil gene or GFP gene, where the uracil gene open reading frame is derived from Saccharomyces cerevisiae, and has a sequence of 5′-ATGCCTGAACTCACCGCGACGTCTGTCGAGAAGTTTCTGATCGAAAAGT TCGACAGCGTCTCCGACCTGATGCAGCTCTCGGAGGGCGAAGAATCTCGT GCTTTCAGCTTCGATGTAGGAGGGCGTGGATATGTCCTGCGGGTAAATAGC TGCGCCGATGGTTTCTACAAAGATCGTTATGTTTATCGGCACTTTGCATCGG CCGCGCTCCCGATTCCGGAAGTGCTTGACATTGGGGAATTCAGCGAGAGCC TGACCTATTGCATCTCCCGCCGTGCACAGGGTGTCACGTTGCAAGACCTGC CTGAAACCGAACTGCCCGCTGTTCTGCAGCCGGTCGCGGAGGCCATGGAT GCGATCGCTGCGGCCGATCTTAGCCAGACGAGCGGGTTCGGCCCATTCGGA CCGCAAGGAATCGGTCAATACACTACATGGCGTGATTTCATATGCGCGATTG CTGATCCCCATGTGTATCACTGGCAAACTGTGATGGACGACACCGTCAGTG CGTCCGTCGCGCAGGCTCTCGATGAGCTGATGCTTTGGGCCGAGGACTGCC CCGAAGTCCGGCACCTCGTGCACGCGGATTTCGGCTCCAACAATGTCCTGA CGGACAATGGCCGCATAACAGCGGTCATTGACTGGAGCGAGGCGATGTTC GGGGATTCCCAATACGAGGTCGCCAACATCTTCTTCTGGAGGCCGTGGTTG GCTTGTATGGAGCAGCAGACGCGCTACTTCGAGCGGAGGCATCCGGAGCT TGCAGGATCGCCGCGGCTCCGGGCGTATATGCTCCGCATTGGTCTTGACCA ACTCTATCAGAGCTTGGTTGACGGCAATTTCGATGATGCAGCTTGGGCGCA GGGTCGATGCGACGCAATCGTCCGATCCGGAGCCGGGACTGTCGGGCGTA CACAAATCGCCCGCAGAAGCGCGGCCGTCTGGACCGATGGCTGTGTAGAA GTACTCGCCGATAGTGGAAACCGACGCCCCAGCACTCGTCCGAGGGCAAA GGAAGGA-3′ (SEQ ID NO: 4).

The rDNA promoter and the rDNA terminator in the exogenous or endogenous gene expression cassette have the following sequences: The rDNA promoter has a sequence of:

(SEQ ID NO: 1)
5′-AGAAAACATAGAATAGTTACCGTTATTGGTAGGAGTGTGGTGGG
GTGGTATAGTCCGCATTGGGATGTTACTTTCCTGTTATGGCATGGAT
TTCCCTTTAGGGTCTCTGAAGCGTATTTCCGTCACCGAAAAAGGCAG
AAAAAGGGAAACTGAAGGGAGGATAGTAGTAAAGTTTGAATGGTGGT
AGTGTAATGTATGATATCCGTTGGTTTTGGTTTCGGTTGTGAAAAGT
TTTTTGGTATGATATTTTGCAAGTAGCATATATTTCTTGTGTGAGAA
GGTATATTTTGTATGTTTTGTATGTTCCCGCGCGTTTCCGTATTTTC
CGCTTCCGCTTCCGCAGTAAAAAATAGTGAGGAACTGGGTTACCCGG
GGCACCTGTCACTTTGGAAAAAAAATATACGCTAAGATTTTTGGAGA
ATAGCTTAAATTGAAGTTTTTCTCGGCGAGAAATACGTAGTTAAGGC
AGAGCGACAGAGAGGGCAAAAGAAAATAAAAGTAAGATTTTAGTTTG
TAATGGGAGGGGGGGTTTAGTCATGGAGTACAAGTGTGAGGAAAAGT
AGTTGGGAGGTACTTCATGCGAAAGCAGTTGAAGACAA-3′

And the rDNA terminator has a sequence of 5′-TTTTTATTTCTTTCTAAGTGGGTACTGGCAGGAGCCGGGGCCTAGTTTAG AGAGAAGTAGACTGAACAAGTCTCTATAAATTTTATTTGTCTTAAGAATTCT ATGATCCGGGTAAAAACATGTATTGTATATATCTATTATAATATACGATGAGGA TGATAGTGTGTAAGAGTGTACCATTTACTAATGTATGTAAGTTACTATTTACT ATTTGGTCTTTTTATTTTTTATTTTTTTTTTTTTTTTCGTTGCAAAGATGGGTT GAAAGAGAAGGGCTTTCACAA-3′ (SEQ ID NO: 2).

The internal ribosome entry site in the exogenous or endogenous gene expression cassette has a sequence of 5′-AAAGCAAAAATGTGATCTTGCTTGTAAATACAATTTTGAGAGGTTAATAA ATTACAAGTAGTGCTATTTTTGTATTTAGGTTAGCTATTTAGCTTTACGTTCC AGGATGCCTAGTGGCAGCCCCACAATATCCAGGAAGCCCTCTCTGCGGTTT TTCAGATTAGGTAGTCGAAAAACCTA-3′ (SEQ ID NO: 3). And the poly(T) sequence in the exogenous or endogenous gene expression cassette has a sequence of 5′-TTTTTTTTTTTTTTTTTTTTTTTTTTTTTTTTTTTTTTTTTTTTTTTTTT-3′ (SEQ ID NO: 5).

The selective marker gene expression cassette may include at least one selective marker gene. The marker gene may be the hygromycin B resistance gene and/or the G418 resistance gene. The hygromycin B resistance gene has a DNA sequence of 5′-ATGTCGAAAGCTACATATAAGGAACGTGCTGCTACTCATCCTAGTCCTGT TGCTGCCAAGCTATTTAATATCATGCACGAAAAGCAAACAAACTTGTGTGC TTCATTGGATGTTCGTACCACCAAGGAATTACTGGAGTTAGTTGAAGCATTA GGTCCCAAAATTTGTTTACTAAAAACACATGTGGATATCTTGACTGATTTTT CCATGGAGGGCACAGTTAAGCCGCTAAAGGCATTATCCGCCAAGTACAATT TTTTACTCTTCGAAGACAGAAAATTTGCTGACATTGGTAATACAGTCAAATT GCAGTACTCTGCGGGTGTATACAGAATAGCAGAATGGGCAGACATTACGAA TGCACACGGTGTGGTGGGCCCAGGTATTGTTAGCGGTTTGAAGCAGGCGG CAGAAGAAGTAACAAAGGAACCTAGAGGCCTTTTGATGTTAGCAGAATTG TCATGCAAGGGCTCCCTATCTACTGGAGAATATACTAAGGGTACTGTTGACA TTGCGAAGAGCGACAAAGATTTTGTTATCGGCTTTATTGCTCAAAGAGACA TGGGTGGAAGAGATGAAGGTTACGATTGGTTGATTATGACACCCGGTGTGG GTTTAGATGACAAGGGAGACGCATTGGGTCAACAGTATAGAACCGTGGATG ATGTGGTCTCTACAGGATCTGACATTATTATTGTTGGAAGAGGACTATTTGC AAAGGGAAGGGATGCTAAGGTAGAGGGTGAACGTTACAGAAAAGCAGGC TGGGAAGCATATTTGAGAAGATGCGGCCAGCAAAACTAA-3′ (SEQ ID NO: 6); and the G418 resistance gene has a DNA sequence of 5′-ATGGGTAAGGAAAAGACTCACGTTTCGAGGCCGCGATTAAATTCCAACA TGGATGCTGATTTATATGGGTATAAATGGGCTCGCGATAATGTCGGGCAATC AGGTGCGACAATCTATCGATTGTATGGGAAGCCCGATGCGCCAGAGTTGTT TCTGAAACATGGCAAAGGTAGCGTTGCCAATGATGTTACAGATGAGATGGT CAGACTAAACTGGCTGACGGAATTTATGCCTCTTCCGACCATCAAGCATTTT ATCCGTACTCCTGATGATGCATGGTTACTCACCACTGCGATCCCCGGCAAAA CAGCATTCCAGGTATTAGAAGAATATCCTGATTCAGGTGAAAATATTGTTGA TGCGCTGGCAGTGTTCCTGCGCCGGTTGCATTCGATTCCTGTTTGTAATTGT CCTTTTAACAGCGATCGCGTATTTCGTCTCGCTCAGGCGCAATCACGAATGA ATAACGGTTTGGTTGATGCGAGTGATTTTGATGACGAGCGTAATGGCTGGC CTGTTGAACAAGTCTGGAAAGAAATGCATAAGCTTTTGCCATTCTCACCGG ATTCAGTCGTCACTCATGGTGATTTCTCACTTGATAACCTTATTTTTGACGA GGGGAAATTAATAGGTTGTATTGATGTTGGACGAGTCGGAATCGCAGACCG ATACCAGGATCTTGCCATCCTATGGAACTGCCTCGGTGAGTTTTCTCCTTCA TTACAGAAACGGCTTTTTCAAAAATATGGTATTGATAATCCTGATATGAATAA ATTGCAGTTTCATTTGATGCTCGATGAGTTTTTCTAA-3′ (SEQ ID NO: 7).

The promoter in the selective marker gene has a sequence of 5′-GACATGGAGGCCCAGAATACCCTCCTTGACAGTCTTGACGTGCGCAGCT CAGGGGCATGATGTGACTGTCGCCCGTACATTTAGCCCATACATCCCCATGT ATAATCATTTGCATCCATACATTTTGATGGCCGCACGGCGCGAAGCAAAAAT TACGGCTCCTCGCTGCAGACCTGCGAGCAGGGAAACGCTCCCCTCACAGA CGCGTTGAATTGTCCCCACGCCGCGCCCCTGTAGAGAAATATAAAAGGTTA GGATTTGCCACTGAGGTTCTTCTTTCATATACTTCCTTTTAAAATCTTGCTAG GATACAGTTCTCACATCACATCCGAACATAAACAACC-3′ (SEQ ID NO: 8). And the terminator in the selective marker gene has a sequence of 5′-ACTGACAATAAAAAGATTCTTGTTTTCAAGAACTTGTCATTTGTATAGTT TTTTTATATTGTAGTTGTTCTATTTTAATCAAATGTTAGCGTGATTTATATTTTT TTTCGCCTCGACATCATCTGCCCAGATGCGAAGTTAAGTGCGCAGAAAGTA ATATCATGCGTCAATCGTATGTGAATGCTGGTCGCTATACTG-3′ (SEQ ID NO: 9).

Another aspect of the present invention provides a method for constructing the above-described expression system. The method includes (1) constructing an expression vector for the Saccharomyces cerevisiae expression system; and (2) expression of exogenous or endogenous gene. Expression of the exogenous or endogenous gene includes inserting a gene coding frame into the expression vector at cleavage sites to be inserted by exogenous genes to obtain a recombinant expression vector; transforming the recombinant expression vector into a host strain Saccharomyces cerevisiae; and screening and verifying positive transformants of the host strain Saccharomyces cerevisiae.

The method for transforming the recombinant expression vector into the host strain Saccharomyces cerevisiae in step (2) may be a PEG-LiAc transformation method. Other methods for transforming the recombinant expression vector into the host strain Saccharomyces cerevisiae in step (2) include electrotransformation and protoplast transformation.

Further aspects of the present invention include a protein expressed by the above expression system. Certain technical effects of the various aspects of the present invention include:

1. Elements including an rDNA promoter, an IRES sequence, a poly (T) sequence, and an rDNA terminator, which are capable of being applied to express an exogenous or endogenous gene expression cassette in Saccharomyces cerevisiae, construct a series of new expression vectors. By using the series of expression vectors, expression of exogenous or endogenous proteins and engineering of metabolic pathways can be conveniently conducted to the yeast.

2. The rDNA promoter in the exogenous gene expression cassette of the present invention can be recognized by and bind to RNA polymerase I, and thus efficient expression of the exogenous gene is completed by means of the efficient function of the RNA polymerase I in the transcription of the rDNA gene.

3. An internal ribosome entry site (IRES) in the exogenous gene expression cassette of the present invention functions as a 5′ cap structure obtained from RNA polymerase II-directed mRNA transcription, and is capable of binding to small ribosome subunits and initiating translation to synthesize proteins, without recruiting any translation initiation factor in vivo.

4. The poly (T) sequence in the exogenous gene expression cassette facilitates transfer of an exogenous gene mRNA from the nucleus to the cytoplasm via a poly(A) tail of 50 bp located at the rear end of the transcribed exogenous gene mRNA, and can enhance the stability of the exogenous gene mRNA.

5. The novel Saccharomyces cerevisiae expression system is also useful in studying the RNA polymerase I-mediated rDNA transcription regulation mechanism and rRNA synthesis mechanism in Saccharomyces cerevisiae.

### BRIEF DESCRIPTION OF THE DRAWINGS

FIG. 1 is a PCR validation diagram of a hygromycin b gene expression cassette constructed onto YEplac195 in Embodiment 1, where M represents a 1 kb DNA marker; and 1 represents a Hyg B-TEF1 terminator gene fragment obtained by amplifying through colony PCR using primers Hyg B-F and pJ-TEF1-Nco I-R;

FIG. 2 is a PCR validation diagram of respective elements of a uracil gene expression cassette constructed onto YEp-Hyg B in Embodiment 1, where M represents a 1 kb DNA marker; and 1 represents a URA3-poly(T)-rDNA terminator gene fragment obtained by amplifying using primers Asc I-URA3-F and rDNAt-Hind III-R;

FIG. 3 is a PCR validation diagram for confirming the transformation of the novel expression vector YEp-Hyg B-RIUTR into Saccharomyces cerevisiae in Embodiment 2, where M represents a 1 kb DNA marker; and 1 represents an rDNA promoter-IRES-URA3 gene fragment obtained by amplifying through PCR using primers Sac I-rDNAp-F and URA3-Xho I-R; and

FIG. 4 shows a gradient growth test of the novel expression system on a synthetic medium with or without uracil in Embodiment 3.

### DETAILED DESCRIPTION

The technical solution of the present invention will be described in detail below with reference to embodiments. These embodiments are for illustrative only, and should not be considered as limiting the scope of the present invention. Modifications or substitutions made to methods, steps or conditions of the present invention are deemed to fall within the scope of the present invention, without departing from the spirit and essence of the present invention.

In order to verify the feasibility and effectiveness of the expression system in a yeast described herein, the expression of a uracil gene is used as an example, and the example in which this expression system is used to express the uracil gene to enable a host strain which cannot synthesize uracil to obtain the ability of synthesizing uracil is illustrated, where the specific implementation process is as follows:

### Embodiment 1: Construction of Yeast Expression Vector

1. Construction of Expression Cassette for Hygromycin B-Resistant Gene

A hygromycin B gene expression cassette Sal I-TEF1p-Hyg B-TEF1t-Nco I which was about 1500 bp and had cleavage sites to be digested by enzymes Sal I and Nco I was obtained through amplification by using the plasmid YEp-CH as a template, and using primers Sal I-pJ-TEF1-F (5′-CATTTCCCCGAAAAGTGCCACCTGACGTCGACATGGAGGCCCAGAATA CC-3′—SEQ ID NO: 10) and pJ-TEF1-Nco I-R (5′-CTTTAGCGGCTTAACTGTGCCCTCCATGGCAGTATAGCGACCAGCATTC AC-3′—SEQ ID NO: 11), where the PCR amplification conditions were 30 cycles of pre-denaturation at 95° C. for 3 min, denaturation at 95° C. for 45 s, annealing at 52° C. for 15 s, and extension at 72° C. for 1.5 min, and final extension at 72° C. for 5 min.

2. Construction of Plasmid YEp-Hyg B Containing Hygromycin B Resistance Gene

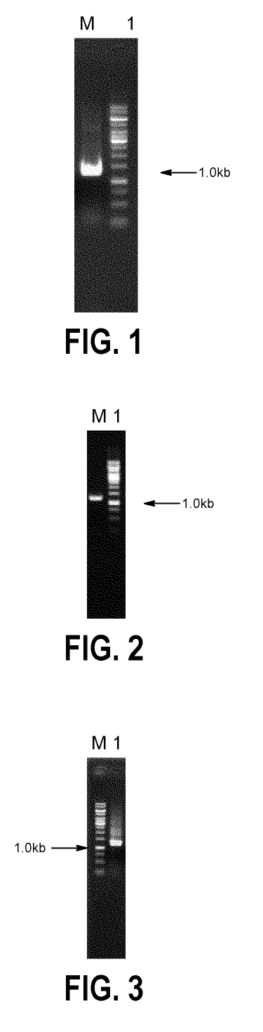

The plasmid YEplac195 and the hygromycin B gene expression cassette Sal I-TEF1p-Hyg B-TEF1t-Nco I were digested by the enzymes Sal I and Nco I, then ligated and transformed into Escherichia coli DH5a. The transformants were picked, and then the plasmid was extracted and validated through colony PCR by using primers Hyg B-F (5′-ATGCCTGAACTCACCGCG-3′—SEQ ID NO: 12) and pJ-TEF1-Nco I-R (5′-CTTTAGCGGCTTAACTGTGCCCTCCATGGCAGTATAGCGACCAGCATTC AC-3′—SEQ ID NO: 11), where the PCR amplification conditions were 30 cycles of pre-denaturation at 95° C. for 3 min, denaturation at 95° C. for 45 s, annealing at 56° C. for 15 s, and extension at 72° C. for 2 min, and final extension at 72° C. for 5 min. A band of about 1300 bp was obtained through the amplification (as shown in FIG. 1), indicating that the hygromycin B expression cassette was successfully ligated onto YEplac195, and a recombinant plasmid YEp-Hyg B containing the hygromycin B gene expression cassette was obtained.

3. Amplification of Respective Elements in Uracil Gene Expression Cassette

(1) Amplification of rDNA promoter: an rDNA promoter fragment which was about 600 bp and had an IRES element homology arm was obtained through PCR amplification by using the genomic DNA of Saccharomyces cerevisiae BY4741 as a template, and using primers Sac I-rDNAp-F (5′-CATTTCCCCGAAAAGTGCCACCTGACGTCGACATGGAGGCCCAGAATA CC-3′—SEQ ID NO: 10) and rDNAp-IRES-R (5′-CTTTAGCGGCTTAACTGTGCCCTCCATGGCAGTATAGCGACCAGCATTC AC-3′—SEQ ID NO: 11), where the PCR amplification conditions were 30 cycles of pre-denaturation at 95° C. for 3 min, denaturation at 95° C. for 45 s, annealing at 52° C. for 15 s, and extension at 72° C. for 40 s, and final extension at 72° C. for 5 min.

(2) Amplification of IRES fragments: The sequence of CrPV intergenic region (IGR) IRES was obtained by looking it up in the NCBI (National Center for Biotechnology Information) Genome. The IRES sequence was then obtained by full-length gene synthesis, and then was subjected to PCR amplification by using a plasmid pUC57-IRES which contains the IRES sequence as a template, and using primers rDNAp-IRES-F (5′-GAAAGCAGTTGAAGACAAGTTCGAAAAGAGAAAGCAAAAATGTGATC TTGC-3′—SEQ ID NO: 13) and Asc I-IRES-R (5′-TTGGCGCGCCTTGAAATGTAGCAGGTAAATTTC-3′—SEQ ID NO: 14), so as to obtain a IRES element fragment which was about 250 bp and had an rDNA promoter element homology arm, where the PCR amplification conditions were 30 cycles of pre-denaturation at 95° C. for 3 min, denaturation at 95° C. for 45 s, annealing at 52° C. for 15 s, and extension at 72° C. for 20 s, and final extension at 72° C. for 5 min.

(3) Fused amplification of rDNA promoter fragment and IRES fragment: a Sac I-rDNAp-IRES-Asc I fragment which was about 850 bp and had cleavage sites to be digested by enzymes Sac I and Asc I was obtained through fused amplification by using the rDNA promoter fragment which had the IRES element homology arm and the IRES element fragment which had the rDNA promoter element homology arm as templates respectively and using primers Sac I-rDNAp-F and Asc I-IRES-R, where the PCR amplification conditions were 30 cycles of pre-denaturation at 95° C. for 3 min, denaturation at 95° C. for 45 s, annealing at 52° C. for 15 s, and extension at 72° C. for 50 s, and final extension at 72° C. for 5 min.

(4) Amplification of uracil gene open reading expression cassette: the uracil sequence was subjected to PCR amplification by for example using a plasmid pJFE3 as a template and using primers Asc I-URA3-F (5′-TTGGCGCGCCATGTCGAAAGCTACATATAAG-3′—SEQ ID NO: 15) and URA3-Xho I-R (5′-CCGCTCGAGTTAGTTTTGCTGGCCGC-3′—SEQ ID NO: 16), so as to obtain a uracil gene open reading frame of about 850 bp, where the PCR amplification conditions were 30 cycles of pre-denaturation at 95° C. for 3 min, denaturation at 95° C. for 45 s, annealing at 52° C. for 15 s, and extension at 72° C. for 50 s, and final extension at 72° C. for 5 min.

(5) Acquisition of poly(T) sequence: Since it was difficult to obtain a poly(T) sequence by PCR, in the present invention the poly(T) sequence were constructed onto the plasmid pUC57-poly(T) through artificial synthesis, and then double digested by enzymes Xho I and Xba I, to obtain a poly(T) containing cleavage sites.

(6) Amplification of rDNA terminator fragment: an rDNA terminator fragment which was about 300 bp and had cleavage sites to be digested by enzymes Xba I and Hind III was obtained through PCR amplification by using the genomic DNA of Saccharomyces cerevisiae BY4741 as a template, and using primers rDNAt-Xba I-F (5′-CTAGTCTAGATTTTTATTTCTTTCTAAGTGGGTAC-3′—SEQ ID NO: 17) and rDNAt-Hind III-R (5′-GATGCTAGCTTGTGAAAGCCCTTCTCTTTC-3—SEQ ID NO: 18), where the PCR amplification conditions were 30 cycles of pre-denaturation at 95° C. for 3 min, denaturation at 95° C. for 45 s, annealing at 50° C. for 15 s, and extension at 72° C. for 25 s, and final extension at 72° C. for 5 min.

4. Construction of Novel Expression Vector YEp-Hyg B-RIUTR

The recombinant plasmid YEp-Hyg B and respective elements in the exogenous gene expression cassette were digested with the corresponding restriction enzymes respectively, then ligated and transformed into Escherichia coli, and then verified accordingly, where after 4 times of ligation and after transformation of Escherichia coli DH5a, the transformants were picked, and then the plasmid was extracted and finally validated through PCR by using primers Asc I-URA3-F and rDNAt-Hind III-R (as shown in FIG. 2), where the PCR amplification conditions were 30 cycles of pre-denaturation at 94° C. for 10 min, denaturation at 94° C. for 30 s, annealing at 52° C. for 30 s, and extension at 72° C. for 1.5 min, and final extension at 72° C. for 10 min. A band of about 1,300 bp was obtained through PCR amplification, indicating that the URA3 open reading frame, the poly(T) and the rDNA terminator were successfully ligated onto YEp-Hyg B, and finally the novel expression vector, i.e., the recombinant plasmid YEp-Hyg B-RIUTR, was obtained. The expression vector contains an hygromycin B resistance gene expression cassette and a uracil gene expression cassette, where the transcription and translation of the uracil gene was achieved by adding the IRES sequence and the poly(T) sequence into the uracil gene under the control of an rDNA promoter and an rDNA terminator.

### Embodiment 2: Construction of the Novel Saccharomyces cerevisiae Expression System

The novel expression vector YEp-Hyg B-RIUTR was transformed into Saccharomyces cerevisiae BY4741 by a transformation method which was a PEG-LiAc-mediated Saccharomyces cerevisiae transformation method. The transformants were screened by a YPD plate containing 200 mg/L hygromycin B, and picked. The plasmids were back-extracted from the yeast, and then subjected to PCR amplification by using primers Sac I-rDNAp-F and URA3-Xho I-R, where the PCR amplification conditions were 30 cycles of pre-denaturation at 95° C. for 3 min, denaturation at 95° C. for 45 s, annealing at 52° C. for 15 s, and extension at 72° C. for 1.5 min, and final extension at 72° C. for 5 min. A band of about 1400 bp was obtained through PCR amplification, indicating that the information expression vector was successfully transformed into Saccharomyces cerevisiae.

### Embodiment 3: Functional Test of the Novel Expression System

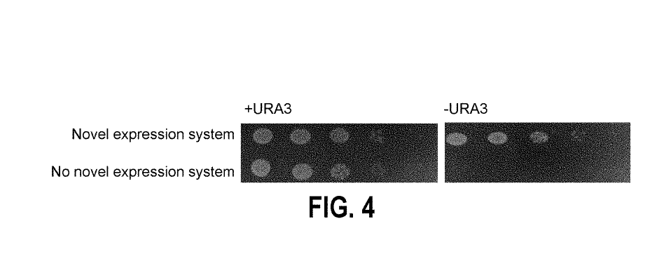

A control strain (i.e., the empty plasmid YEp-Hyg B not containing the URA3 gene expression cassette) and an experimental strain (i.e., Single colony of Saccharomyces cerevisiae which expressed the uracil gene (containing the plasmid YEp-Hyg B-RIUTR) which had been subjected to plate streaking were picked and inoculated into YPD, and then subjected to activated shaking culture at 30° C. twice. The strains were cultured overnight, taken out at a late stage of the logarithmic growth phase, collected by centrifugation, washed with sterile water for three times, re-suspended in 1 mL sterile water, incubated in an incubator at 30° C. for 9 h to consume endogenous nutrients, so as to prepare resting cells. The resting cell concentration of the strains was regulated to achieve a suspension OD₆₀₀ of about 1, and 10-fold serially diluted to three dilutions (10⁰, 10⁻¹, 10⁻², and 10⁻³). 4 μL of the diluent was dropped onto a synthetic medium plate containing or not containing uracil, and cultured thereon at 30° C. for 3-5 days to observe the colony growth condition. The results were photographed and as shown in FIG. 4, where both the control strains and the experimental strains grew well on the synthetic medium containing uracil; and the control could not grow on the synthetic medium not containing uracil since it could not synthesize uracil due to the lack of the uracil gene expression cassette, while the experimental strain which expressed the uracil gene using the novel expression vector could grow relatively well on the synthetic medium not containing uracil, which indicates that under the action of the RNA polymerase I, the uracil gene was transcribed under the control of the rDNA promoter and the rDNA terminator, and was successfully translated into uracil under the action of the Cricket paralysis virus intergenic region (CrPV IGR) IRES sequence and the poly(T), such that the experimental strain can eventually grow on the medium not containing uracil.

The embodiments described above are only descriptions of preferred embodiments of the present invention, and do not intended to limit the scope of the present invention. Various variations and modifications can be made to the technical solution of the present invention by those of ordinary skills in the art, without departing from the design and spirit of the present invention. The variations and modifications should all fall within the claimed scope defined by the claims of the present invention.

What is claimed is:

1. A Saccharomyces cerevisiae expression system consisting of a host transfected by an expression vector, wherein the expression vector is circular and is a shuttle plasmid vector constructed between Saccharomyces cerevisiae and Escherichia coli, and the expession vector comprises a plurality of operable elements, the plurality of operable elements comprising sequentially from 5′ to 3′ a YEplac195 plasmid backbone, an exogenous or endogenous gene expression cassette, and a selective marker gene expression cassette;
wherein the YEplac195 plasmid is a yeast episomal plasmid including an ori of a yeast 2μ plasmid;
wherein the exogenous or endogenous gene expression cassette comprises sequentially from upstream to downstream: an rDNA promoter, an internal ribosome entry site sequence, an exogenous or endogenous gene expression cassette, a poly(T) sequence, and an rDNA terminator; and
wherein the selective marker gene expression cassette comprises a promoter, a selective marker gene, and a transcription terminator.

2. The expression system of claim 1, wherein the exogenous or endogenous gene in the exogenous or endogenous gene expression cassette is a uracil gene or GFP gene.

3. The expression system of claim 2, wherein the uracil gene is derived from Saccharomyces cerevisiae, and has a sequence of SEQ ID NO: 4.

4. The expression system of claim 1, wherein the rDNA promoter and the rDNA terminator in the exogenous or endogenous gene expression cassette have sequences of SEQ ID NO: 1 and SEQ ID NO: 2 respectively;
the internal ribosome entry site sequence in the exogenous or endogenous gene expression cassette is SEQ ID NO: 3; and
the poly(T) sequence in the exogenous or endogenous gene expression cassette is SEQ ID NO: 5.

5. The expression system of claim 1, wherein the sequence of the promoter in the selective marker gene expression cassette is SEQ ID NO: 8; and the sequence of the terminator in the selective marker gene expression cassette is SEQ ID NO: 9.

6. The expression system of claim 5, wherein the selective marker gene in the selective marker gene expression cassette is a hygromycin B resistance gene and/or a G418 resistance gene; wherein the sequence of the hygromycin B resistance gene is SEQ ID NO: 6, and the sequence of the G418 resistance gene is SEQ ID NO: 7.

7. A method for constructing an expression system comprising:
constructing the expression vector of claim 1;
inserting a gene coding frame into the expression vector at cleavage sites to be inserted by exogenous genes to obtain a recombinant expression vector;
transforming the recombinant expression vector into a host strain Saccharomyces cerevisiae; and
screening and verifying positive transformants of the host strain Saccharomyces cerevisiae.

8. The method of claim 7, wherein transforming the recombinant expression vector into the host strain Saccharomyces cerevisiae comprises PEG-LiAc transformation, electrotransformation, or protoplast transformation.

9. A protein expressed by the expression system of claim 1.
